(19) United States
(12) Patent Application Publication
(10) Pub. No.: US 2019/0014982 A1
(43) Pub. Date: Jan. 17, 2019

(54) AUTOMATED BLOOD VESSEL FEATURE DETECTION AND QUANTIFICATION FOR RETINAL IMAGE GRADING AND DISEASE SCREENING

(71) Applicant: iHealthScreen Inc., Charlottesville, VA (US)

(72) Inventor: Mohammed Alauddin Bhuiyan, Charlottesville, VA (US)

(73) Assignee: iHealthScreen Inc., Charlottesville, VA (US)

(21) Appl. No.: 15/647,430

(22) Filed: Jul. 12, 2017

(51) Int. Cl. A61B 3/00; A61B 3/12; G06T 7/60; G06T 7/187; G06K 9/00; G06T 7/13; G06T 7/44; G06K 9/62

(52) U.S. Cl. CPC A61B 3/0025; A61B 3/1241; G06T 7/60; G06T 7/187; G06K 9/00617; G06K 9/0061; G06T 2207/20061; G06T 7/44; G06K 9/6223; G06T 2207/30041; G06T 2207/30101; G06T 2207/20044; G06T 7/13

(57) ABSTRACT

A method for vessel mapping and quantification. The method comprises pre-processing a retinal image to generate a vessel segmented image and processing the vessel segmented image to generate an image with a central light reflex. The method further includes identifying a cylindrical or tube-shaped region in the central light reflex and determining a closed contour representing the cylindrical shaped region and representing the closed contour by a function. The method computes a ratio of a minimum and maximum radius of the cylinder to determine an average shape of the cylinder associated with the central light reflex by using the function. The image is further processed to apply a morphological skeletonization operation to generate vessel centerlines and the segmented vascular network of the retinal image. In an example embodiment, a method for artery-vein nicking quantification for retinal blood vessels is performed by computing width of the vessel near and away from a cross over point of the vessels. In another example embodiment, a feature associate with the central light reflex is identified and compared with a second feature in the same location evaluated at a different time zone to confirm the associated shape of the light reflex. In another example embodiment, retinal focal arteriolar narrowing (FAN) is identified and quantified value is generated. In another example embodiment, a true optic disc is identified based on a combination of features and parameters associate with the vessel.

### CROSS REFERENCE TO RELATED APPLICATION

Not Applicable

### FIELD

The disclosure relates to a number of methods for detecting and quantifying retinal vessel features using retinal image which can be used to screen retinal and systemic diseases.

### BACKGROUND

Research suggests that retinal vessel features are associated with many systemic diseases such as Cardiovascular Heart Disease (CVD), Stroke and Alzheimer's or Dementia, and can be used as precursor of these diseases (Please see non-patent literature—Recent Patents on Computer Science 2010, 3(3); 164-175, by Bhuiyan et al., Lancet 2001, 358(9288):1134-1140, by Wong et al., and Transl Psychiatry 2013, 3, e233; doi:10.1038/tp.2012.150:1-8, by Frost et al., For instance, arteriolar narrowing (measured from width) is associated with stroke, coronary heart disease, hypertension and diabetes (Please see Recent Patents on Computer Science 2010, 3(3):164-175, by Bhuiyan et al.). Venular widening is associated with stroke, heart disease, diabetes and obesity. Retinal vessel caliber is also independently associated with risk of 10-year incident nephropathy, lower extremity amputation, and stroke mortality in persons with type 2 diabetes (Please see Recent Patents on Computer Science 2010, 3(3):164-175, by Bhuiyan et al., Lancet 2001, 358(9288):1134-1140, by Wong et al). Retinal Arteriolar tortuosity is associated with hypertension and diabetes (Please see Recent Patents on Computer Science 2010, 3(3):164-175, by Bhuiyan et al.). Enhanced arteriolar wall reflex is associated with hypertension and Alzheimer's disease (Please see New Engl Journal Med 2004, 351 (22) 2310-2317 by Wong et al.). Arteriovenous Nicking (AVN) is associated with hypertension and stroke (Please see Recent Patents on Computer Science 2010, 3(3):164-175, by Bhuiyan et al.). Focal Arteriolar Narrowing (FAN) is associated with hypertension and stroke (Please see Recent Patents on Computer Science 2010, 3(3):164-175, by Bhuiyan et al.). New vessel formation is associated with Late AMD (Please see Prog Retin Eye Res 2014, 38(doi: 10.1016/j.preteyeres.2013.10.002):20-42, Kanagasingam et al.). DR causes the widening of venular diameter symptoms in its early stage, ROP and plus diseases cause more arterial tortuosity and more venous dilatation than normal (Please see Arch Ophthalmol 2011, 130(6): doi:10.1001/archophthalmol.2011.2560:749-755, Klein et al.).

The most common trends in the analysis of the retinal vascular network are manual examination and semiautomatic methods, which are time-consuming, costly, prone to

inconsistencies and subject to human error. For example, the width measured manually or semi-automatically varies from one inspection to the next, even when the same grader is involved.

Although several research articles have appeared on retinal vessel feature analysis, in particular for vessel width/caliber measurement, tortuosity analysis, branch/bifurcation angle measurement, AV nicking and FAN quantification, automatic analysis of these features is still an open area for improvement. Most of the techniques are semi-automatic and require expert intervention. Repeatability and reliability are mostly compromised with manual or semiautomatic method.

Automatic feature analysis starts with vessel segmentation and vessel classification (i.e. a vessel as artery or vein). A large number of vessel segmentation methods have been proposed which are based on color retinal imaging. However, most of them have failed because they are unable to generate or identify individual vessel-segment with high accuracy which is a significant aspect of automatic feature analysis. Another important factor for feature analysis is to classify vessels as artery and vein. Only a small number of methods have been developed for artery-vein classification. Most of the techniques are unable to produce accurate results for high-resolution images when the vessel light reflex and other pathologies are present. For this reason, to date, vessel feature analysis methods such as vessel width measurement, tortuosity computation, branch angle computation, central reflex quantification, focal arteriolar narrowing and AVN quantification are mostly manual or qualitative or semi-automatic.

Any discussion of the prior art throughout the specification should in no way be considered as an admission that such prior art is widely known or forms part of the common general knowledge in the field.

In this specification, the terms “comprises”, “comprising” or similar terms are intended to mean a non-exclusive inclusion, such that a methods system or apparatus that comprises a list of elements does not include those elements solely, but may well include other elements not listed.

### SUMMARY

The disclosure is directed to methods of detecting and quantifying retinal blood vessel features. The features are detected and quantified by one or more the methods, the methods comprise: retinal blood vessel segmentation, artery-vein classification, vessel centerline detection, optic disc detection, optic disc center detection, optic disc radius computation, vessel edge detection, vessel central reflex detection and vessel segments hierarchical position tracking.

According to an example embodiment of the inventive aspect a method for vessel classification comprises: pre-processing a retinal image to generate a vessel segmented image; first processing the vessel segmented image to generate an image with a central light reflex; identifying a cylindrical shaped region in the central light reflex; determining a closed contour representing the cylindrical shaped region and representing the closed contour by a function; computing a ratio of a minimum and maximum radius of the cylindrical shape of the central light reflex to determine an average shape of the cylinder associated with the central light reflex by using the function; performing region filling for filling at least a hole in vessel center, the hole being formed due to central light reflex; second processing the image to apply a morphological skeletonization operation to generate vessel centerlines and segmented vascular network of the retinal image.

According to another example embodiment of the inventive aspect a method of optic disc detection from a retinal image comprises processing the retinal image to generate a vessel segmented image to identify blood vessels; determining approximate optic disc centers based on a first information associated with the retinal image and the first set of a parameter associated with the vessels; processing the retinal image to determine a plurality of shifted optic disc centers from among the approximate optic disc centers; filtering the shifted optic disc centers based on a criterion and selecting a number of optic disc centers and determining an average of the selected optic disc centers to obtain a first optic disc center; performing Hough transformation using the first optic disc center to determine a first optic disc; determining a second set of parameters associated the first optic disc center and the first optic disc; and using a combination of the second set of parameters to identify a true optic disc (OD).

According to another example embodiment of the inventive aspect a method for retinal blood vessel feature quantification comprises: processing a retinal image to generate vessel centerline; generating a vessel segmented image of a retinal image to identify blood vessels; and further comprising classifying the-arteries or veins based on a combination of a first parameter and a second parameter

According to yet another example embodiment of the inventive aspect a computer implemented system for vessel segmentation of a retinal image comprises a processor and a memory, wherein the memory comprises a non-transitory computer-readable-medium having computer-executable instructions stored therein that, when executed by the processor, cause the processor to: pre-process the retinal image and store the pre-processed image in the memory; perform texture analysis using a Gabor filtering module; perform Otsu's clustering to cluster or segment vessel pixels of the texture analyzed image; detect one or more central light reflexes by identifying a shaped region; and perform region filling to generate a vessel segmented image of the retinal image.

According an example embodiment a method for automated retinal image quantification from a remote location comprises: taking a fundus image from a patient; storing the fundus image and the associated patient information; de-identifying the patient information using an encryption algorithm and security key and sending the encrypted patient information along with the images to a server; calling a software module stored in the server to evaluate and grade the fundus image; selecting an image grader from a list of authorized image grader stored in the server to inspect and finalize features and grading quality of the image, the image grader being selected based on a location information and availability of the image grader; using the security keys to decode and open the patient information associated with the fundus image in the server at the image grader side; in response to an authorization of the fundus image by the image graders, automatically examining, by a software module, and quantifying a plurality of feature values in the fundus image; comparing the quantified features values with a plurality of cutoff values or threshold values associated with specific diseases; and generating a report recommending a referral to an expert based on the comparison.

According an example embodiment an online system for facilitating image transfer from a remote capturing to image grading system and generating an alert to the remote healthcare worker to further advise to a patient, comprises: a digital fundus camera configured to capture a retinal image; a server, comprising: a processor and a memory, wherein the memory comprises a non-transitory computer-readable-medium having computer-executable instructions stored therein that, when executed by the processor, causes the processor to:
store the retinal image and the associated patient information into the server; call a first software module stored in the server to evaluate and grade the fundus image; select an image grader from a list of authorized image grader stored in the server to inspect and finalize features and grading the quality of the fundus image, the image grader being selected based on a location information and availability of the image grader; in response to receiving an authorization of the fundus image by the image graders, call a second software module to automatically examine and quantify a plurality of feature values in the Hindus image; call an evaluating and grading algorithm stored in the server to compare the quantified features values with a plurality of cutoff or threshold values associated with specific diseases; and generate a report recommending a referral to an expert based on the comparison.

In an example embodiment, a method for segmenting the blood vessels in a retinal image includes the steps of:
Image normalization, Gabor filtering for texture analysis and then unsupervised clustering algorithm, i.e., Otsu's method to segment the blood vessels and following this region growing and vessel mapping for identifying the blood vessels. The vessel segmentation method aims to extract the retinal vascular network from the background in the image.

In another example embodiment, a method for optic disc detection and center computation in a retinal image includes the steps of:

At first, the method automatically determines the threshold intensity value by approximating the OD area information in each image. Following this, Region Growing technique is applied in the threshold intensity to identify the potential OD regions. Then, the canny edge detection, Hough circle detection algorithm, vessel centerline detection, vessel segments alignment or slope information, vessel segment length, vessel segment numbers and vessel branch-point density are computed to identify the OD region and compute its center and radius.

In an example of embodiment cup-disc ratio measurement includes a method of detecting cup area by applying Otsu's clustering method to cluster the pixels within the disc area/boundary. Then their ratio is computed by finding the total number of the disc and cup pixels.

In yet another example embodiment, a method for classification of the vessels into artery and vein in a retinal image broadly includes the steps of:
Vessel centerline detection and vessel branch and crossover point computation, vessel crossing mapping, individual vessel-segment's centerline identification, vessel-segment hierarchy determination and individual vessel-segment's width measurement, vessel's color and intensity matrix generation, vessel central light reflex (CR) width determination, vessel position determination and finally, classification of vessel based on computing the vessel location (by angle front OD center) in the image, width, intensity matrix, artery-vein crossing information, CR width and neighborhood vessel class information (i.e., neighbor is artery or vein).

In yet another example embodiment, inventive aspect include the feature computation and quantification.

The features such as vessel-segment (individual artery segment and vein segment) width, vessel width-to-CR width ratio, vessel segments' tortuosity, focal narrowing, artery-vein nicking and branch angle are computed based on the vessel centerline, edge, intensity and width information. The features for branch angle and AVN are computed from the branch and crossover regions.

For vessel-segment width measurement: the individual vessel centerline is used to find the slope and select the vessel area, and to crop a square shaped region. Then vessel edges are identified. Then a bi-pair relation is created by pairing the opposite edge pixels, taking the position of the edge pixel in the edge, and pairing it with with all edge pixels from the opposite edge, and so on (mapped through all different permutations). Then the shortest distance between the opposite edge pixels' pairs is computed for finding the individual vessel cross-sectional width.

Vessel width-to-CR width ratio measurement: The central reflex (CR) for each of the major vessel-segments in the segmented image is identified. If CR is present, the central light reflex edges are identified and the width of central light reflex is computed utilizing the same technique as described in vessel-segment width measurement. Then, the ratio of central light reflex width and vessel width is computed.

Vessel segments' tortuosity measurement: For each vessel segment centerline, the curvature and simple tortuosity and normalize the tortuosity by multiplying the normalized vessel width is computed.

Focal arteriolar narrowing (FAN) quantification: The vessel segment is divided into a number of small-segments within 5 to 10 cross-sections/cross-sectional-widths. Then each of these arterial segments is considered for finding the FAN. The average width for individual small segment is computed. Then the ratio of the cross-sectional widths of the small-segments (width of the small segment/width of the largest segment) is computed. The ratio is than compared with a predetermined threshold value based on the retinal image dataset used to find the FAN.

Artery-vein nicking (AVN) quantification: For the crossover region, the vein segments are selected, and cross-sectional widths are considered for AVN quantification. At first, the artery vein is divided into four segments by utilizing the crossover point. Then artery and vein classification information is used to determine the AVN location and value. The mean width of each of the vein-segments is computed by finding the mid-point of the segment and total ten cross-sections are selected from each of the two sides of the middle of the segment and starting from mid-point+5 pixels. Then the ratio of the width for ten cross-sections closer to the crossover point, and the mean width which is the AVN for a threshold intensity is measured.

Branch angle computation: Vessel hierarchy is generated by traversing from outside the optic disc and traversing through the vessel centerline. Each time a branch point is identified where a specified length of three vessel segments exists. For each branch point in the hierarchy, the branch angle is computed for the angle between the children vessel segments.

In yet another example embodiment, the inventive aspect include the feature computation and quantification in the Zone B and Zone C area or for the individual vessel segments.

Zone B and Zone C area are computed based on the OD center and radius (FIG. 3). Zone B is the circular region which, starts at 1× Optic-Disc Diameter and ends at 1.5× Optic-Disc Diameter from the Optic-Disc-center in the retinal image. Zone C is the rest of the image area outside zone B.

The vessel segments are identified/of zone B and Zone C, and features are mapped for these vessel segments.

Similarly, the features are computed and represented for individual vessel-segments with the hierarchical position, vessel number (to specify which vessel by positional information around the optic disc) and classification (i.e., artery or vein) information.

### An Online Image Grading Platform and Automated Report Generation

In a further implementation, an online grading system configured to facilitate image transfer from a remote image and data capturing system, and configured to grading image and finally reporting is provided. The system generates reports based on cutoff values and alerts or refers to the specialist doctors through remote health care worker to further advise to the patient. At first, remote health provider uses the retinal image collection system/interlace and an eye camera to take fundus images from a patient. Then the healthcare provider uses a web browser application to login into a server system and inputs the patient data and associated disease history and upload the image(s) into the server. The server side module/program stores the patient information into the server's database and automatically calls a software to grade the image. An image grader will be automatically assigned based on the availability to decide about the grading quality, check and finalize the feature gradings. Then a decision support system (i.e., software module) automatically examines the quantified feature values and compares the cutoff values associated with specific diseases. For example, the system checks the artery and vein widths and compares the values for the risk of cardio vascular diseases. Then, the system automatically generates a report for the healthcare worker as well as patient with a referral to an ophthalmologist or an expert Doctor/Consultant.

### BRIEF DESCRIPTION OF THE DRAWINGS

The example embodiments of the disclosure will be better understood from the following brief description taken in conjunction with the accompanying drawings. The drawing FIGS. 1-25 represent non-limiting, example embodiments.

FIG. 1 is a retinal color image showing the blood vessels: arteries and veins, and optic disc and cup according to an example embodiment.

FIG. 2A shows the vessel widths with the black lines and the central light reflex (black arrow) in the vessel according to an example embodiment.

FIG. 2B shows the vessel tortuosity (yellow curve) according to an example embodiment.

FIG. 2C shows the branch angle (yellow color) according to an example embodiment.

FIG. 2D shows the focal arteriolar narrowing within the circular region according to an example embodiment.

FIG. 2E shows the arteriovenous nicking pointed by the black arrow according to an example embodiment.

FIG. 2F shows normal crossover according to an example embodiment.

FIG. 3 shows the different zones in the retinal image—zone A, zone B and zone C according to an example embodiment.

FIG. 4 shows the overall flow diagram of the methods for feature computation according to an example embodiment.

FIG. 5A shows an input color image for the segmentation method according to an example embodiment.

FIG. 5B shows vessel, segmented output image according to an example embodiment.

FIG. 6A shows the centroid to boundary distance approach according to an example embodiment.

FIG. 6B shows the output image showing a feature associated with the central reflex effect according to an example embodiment.

FIG. 6C shows the output image after region filling operation according to an example embodiment.

FIG. 7A shows a retinal color image according to an example embodiment.

FIG. 7B shows a vessel segmented image according to an example embodiment.

FIG. 7C shows a vessel centerline image according to an example embodiment,

FIG. 8 shows the overall method for optic disc detection according to an example embodiment.

FIGS. 9A-9D show the vessel crossover mapping through individual vessel centerline identification according to an example embodiment.

FIG. 10 shows the vessel edge or boundary in zone B area according to an example embodiment.

FIG. 11 shows the mapping of the points on left side (Lₓ;L_{y}) and right side (Rₓ;R_{y}) of an edge pixel (x₂; y₂) according to an example embodiment.

FIGS. 12A-12D show the mapping of vessel and background pixels for intensity profiling according to an example embodiment.

FIGS. 13A-13D show some of the steps for artery-vein classification according to an example embodiment.

FIG. 13A shows all vessel boundary according to an example embodiment.

FIG. 13B shows the classified arteries (red) and veins (blue) based on vessel crossover only (green as unclassified) according to an example embodiment.

FIG. 13C shows the classified arteries and veins based on the widest vessel and classified neighbor(s) according to an example embodiment.

FIG. 13D shows all major vessels classified as arteries and veins according to an example embodiment.

FIG. 14A shows a cropped image showing blood vessel according to an example embodiment.

FIG. 14B shows measured cross-sectional width of the cropped image according to an example embodiment.

FIG. 15A shows the central light reflex in an artery according to an example embodiment.

FIG. 15B shows the competed widths of central light reflex (in blue) and vessel widths (in white) according to an example embodiment.

FIG. 16A shows a presence of focal narrowing in an artery according to an example embodiment.

FIG. 16B shows the measured widths for the segments for finding the focal arteriolar narrowing location and quantification according to an example embodiment.

FIG. 17 shows the vein widths (white lines) for AV nicking quantification according to an example embodiment.

FIG. 18A shows a potential Optic Disc region according to an example embodiment.

FIG. 18B shows the detected circular region of the optic-disc after applying Hough transformation according to an example embodiment.

FIG. 19A shows a retinal color image according to an example embodiment.

FIG. 19B shows the optic-disc boundary marked as red and cup boundary marked as blue according to an example embodiment.

FIG. 20 shows interconnection system and apparatus for image capturing and online grading system according to an example embodiment.

FIG. 21 shows interconnection system and apparatus at an image capturing station according to an example embodiment.

FIG. 22 shows system components and interconnection at the image grading station of the system according to an example embodiment.

FIGS. 23-24 show method-steps involved in comparing central light reflex features/shape associated with a vessel of a retinal image according to an example embodiment.

FIGS. 25A-25D show feature (such as area) comparison associated with a central light reflex according to an example embodiment.

Skilled addressees will appreciate that elements in at least some of the drawings are illustrated for simplicity and clarity and have not necessarily been drawn to scale. For example, the relative dimensions of some of the elements in the drawings may be adjusted to help improve understanding of embodiments of the present disclosure.

### DETAILED DESCRIPTION OF THE DISCLOSURE

In the following description, reference is made to the accompanying drawings which form a part hereof and in which is shown by way of illustration specific embodiments in which the inventive concept may be practiced. These embodiments are described in sufficient detail to enable those skilled in the art to practice the inventive concept, and it is to be understood that the embodiments may be combined, or that other embodiments may be utilized and that structural and logical changes may be made without departing from the spirit and scope of the present inventive concept. The following description is, therefore, not to be taken in a limiting sense.

Example embodiments are described many different forms and should not be construed as being limited to the embodiments set forth herein; rather, these embodiments are provided so that this disclosure will be thorough and complete, and will fully convey the concept of example embodiments to those of ordinary skill in the art. In the drawings, some dimensions may be exaggerated for clarity.

It will be understood that when an element is referred to as being, “connected” or “coupled” to another element, it can be directly connected or coupled to the other element or intervening elements may be present.

The terminology used herein is for the purpose of describing particular embodiments only and is not intended to be limiting of example embodiments of the inventive concepts. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly-understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

The disclosure relates to methods of detecting the optic disc (OD), blood vessels, classifying vessels as artery and vein, measuring vessel caliber/width, quantifying arteriolar central reflex 203, focal narrowing 206, Artery-vein nicking (AVN) 207, width-normalized-tortuosity and hierarchical-branch/bifurcation angle 205 and measuring the OD center, OD radius OD cup-disc ratio. The disclosure has application in systemic and epidemiological diagnosis such as Diabetes, Heart disease, Stroke and Alzheimer's disease. The disclosure can also be used for early screening of age-related macular degeneration (AMD), diabetic retinopathy (DR) and retinopathy of prematurity (ROP).

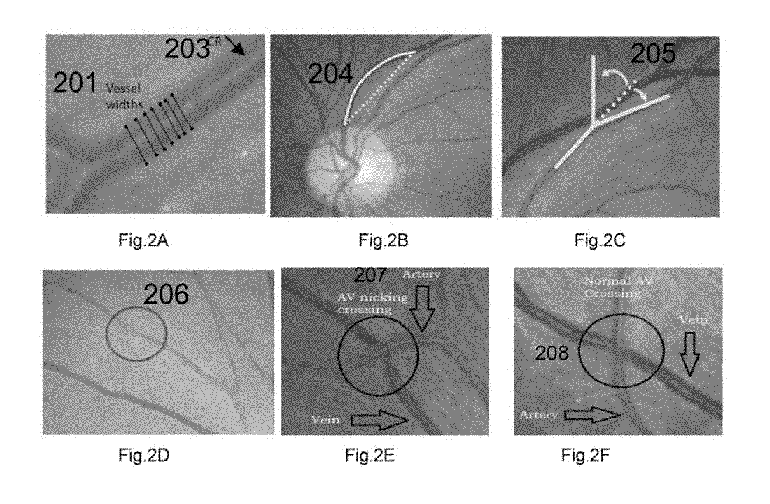

The disclosure relates, at least in part, to methods for detecting features in a retinal image. The present disclosure provides novel and inventive methods for detecting or segmenting the blood vessels, identifying the vessel central reflex, optic disc, and measuring the optic disc center, optic disc radius, the classification of vessels into artery and vein by utilizing vessel mapping and hierarchical information, computing features such as vessel caliber/width, vessel central light reflex quantification, width normalized tortuosity, focal arteriolar narrowing, AV nicking and branch angle. FIG. 2 shows these features according to an example embodiment.

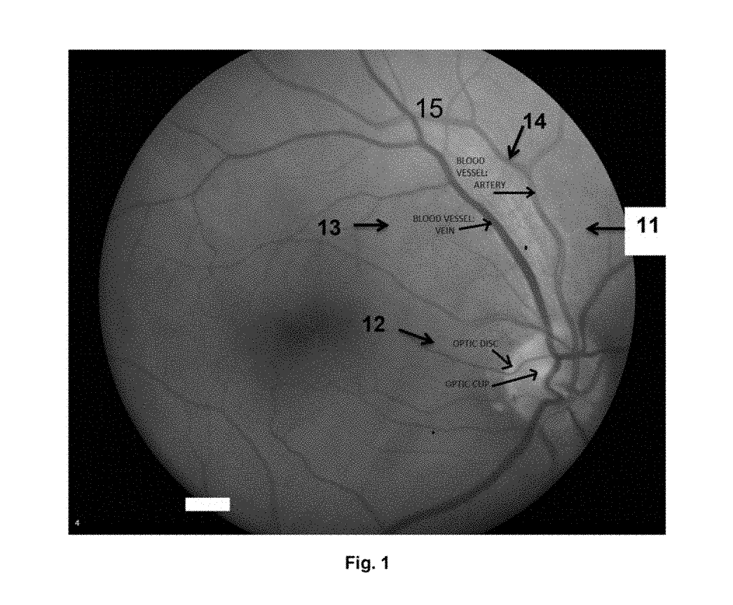

FIG. 1 shows a retinal color image showing the blood vessels: artery 14 and vein 13, and optic disc 12 and cup 15 according to an example embodiment.

FIG. 2A shows the vessel widths with the black lines 201 and the central light reflex 203 (black arrow) in the vessel according to an example embodiment. FIG. 2B shows the vessel tortuosity 204 (yellow curve) according to an example embodiment. FIG. 2C shows the branch angle 205 (yellow color) according to an example embodiment. FIG. 2D shows the focal arteriolar narrowing 206 within the circular region according to an example embodiment. FIG. 2E shows the arteriovenous nicking 207 indicated by a circle, according to an example embodiment. FIG. 2F shows normal crossover 208 indicated by a circle according to an example embodiment.

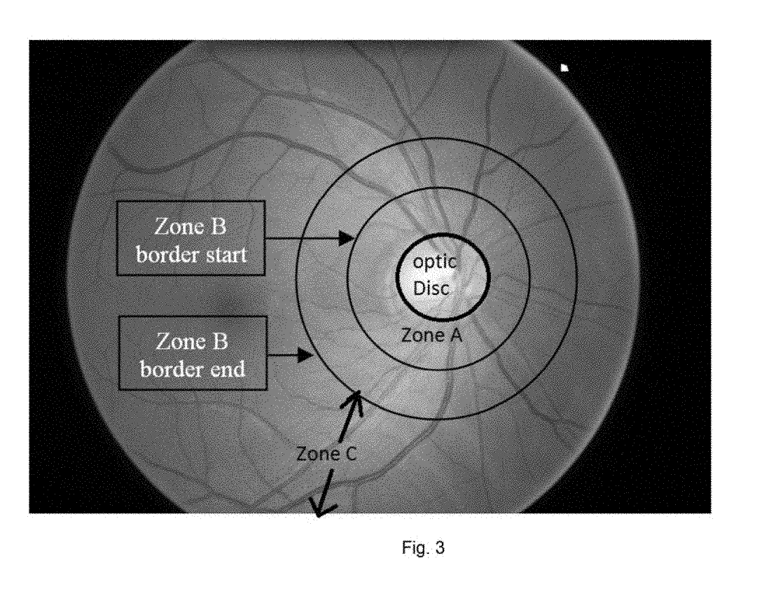

FIG. 3 shows the different zones in the retinal image—zone A, zone B and zone C according to an example embodiment.

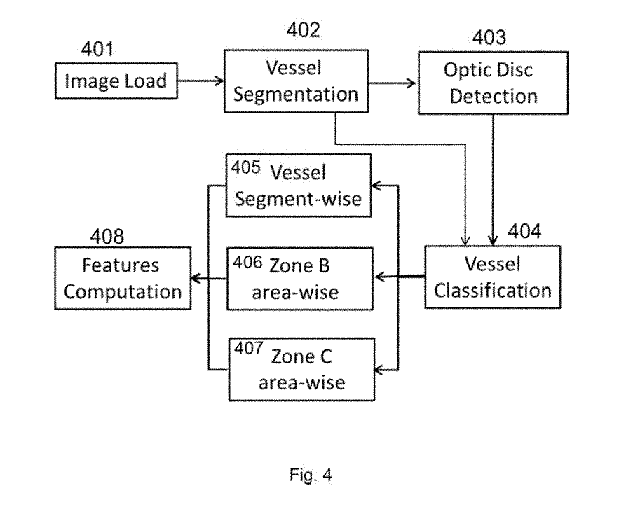

FIG. 4 shows the overall flow diagram describing steps for feature computation according to an example embodiment. In step 401 a retinal image is loaded, and the image is processed for vessel segmentation which is followed by optic disc detection 403. At step 404, vessel classification is performed. The vessel classification may be done by vessel segment wise (step 405), zone B wise (step 406), and Zone C-wise (step 407). In step 408, features are computed.

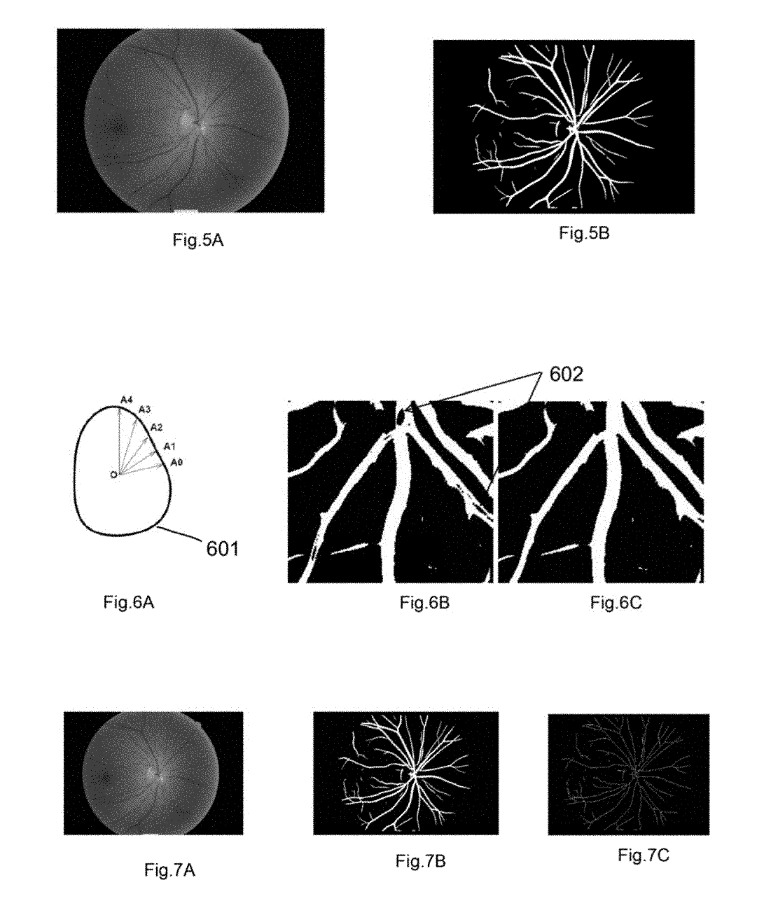

FIG. 5A shows an input color image for the segmentation method and FIG. 5B shows vessel segmented output image according to an example embodiment.

FIG. 6A shows the centroid (O) to boundary distance approach according to an example embodiment. First, a cylindrical or tube-shaped region 601 is identified in the central light reflex. A closed contour representing the shaped region is determined, and is represented by a 1D complex function. For the identified region, centroid O to boundary distance A₀, A₁, A₂, A₃ and A₄ is calculated. A ratio of the minimum and maximum radius of the cylinder is determined to obtain the average shape of the cylinder associated with the central light reflex. Next, morphological skeletonization operation is performed to generate vessel centerlines. Finally, the vessel centerline image is processed to generate the segmented vascular network of the retinal image.

FIG. 6B shows the output image showing central reflex effect according to an example embodiment. FIG. 6C shows the output image after region filling operation according to an example embodiment.

FIG. 7A shows a retinal color image according to an example embodiment FIG. 7B shows a vessel segmented image according to an example embodiment. FIG. 7C shows vessel centerline image according to an example embodiment.

### Texture Analysis by Gabor Filtering

### Preprocessing and Image Normalization

Following a Median filter to remove occasional salt-and-pepper noise, the image is further smoothed by convolving the image with a Gaussian filter. Region growing and feature calculation are made more reliable by this step. The background intensity is estimated by applying 11×11 (it can be 5×5 or 7×7 or 11×11 or 13×13 or up to 25×25 based on the image resolution) median filter. A shade corrected image is generated with S′I/B−1 and is normalized for global image contrast by dividing its standard deviation S=S′/std(S′).

### Gabor Filtering

Texture generally describes second order property of surfaces and scenes, measured over image intensities. A Gabor filter has weak responses along all orientations on the smooth (background) surface. On the other hand, when it is positioned on a linear pattern object (like a vessel) the Gabor filter produces relatively large differences in its responses when the orientation parameter changes. Hence, the use of Gabor filters to analyze the texture of the retinal images is very desirable.

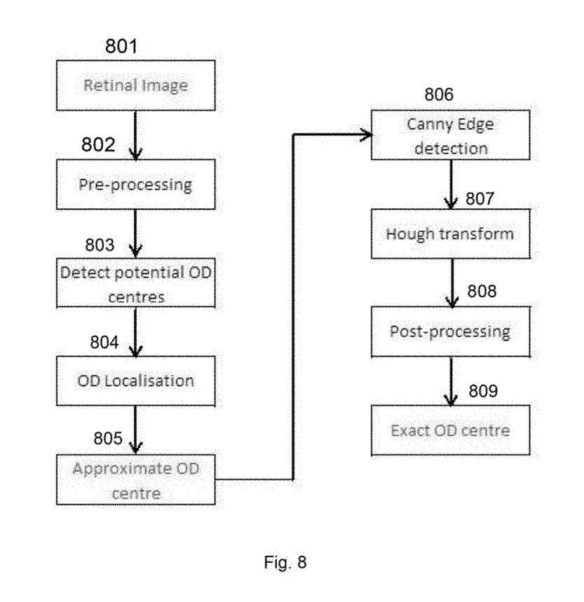

FIG. 8 shows the steps for optic disc detection according to an example embodiment. In step 802, a retinal image is preprocessed. The preprocessing includes normalizing the intensity of the retinal image; applying Gabor filters to the normalized image for edge detection, and processing the normalized image by Otsu's method after Gabor filtering to generate a vessel segmented image.

Following this, the vessel map is obtained for individual vessel center line identification. The individual vessel is classified as artery and vein, and its related features are computed. The vessel map is obtained by searching the vessel around the optic disc. The method for optic disc detection is as follows.

### Optic Disc Detection

Optic disc (OD) center is computed to map the vessel centerlines and to find the zone B and zone C areas. OD is also used to find the vessel location and position information for classifying artery and vein.

The proposed method utilizes OD size, shape and color information and vessel density to detect OD.

### OD Detection

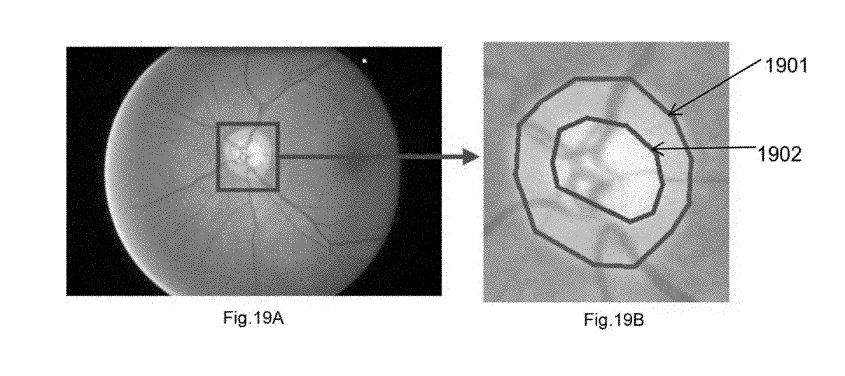

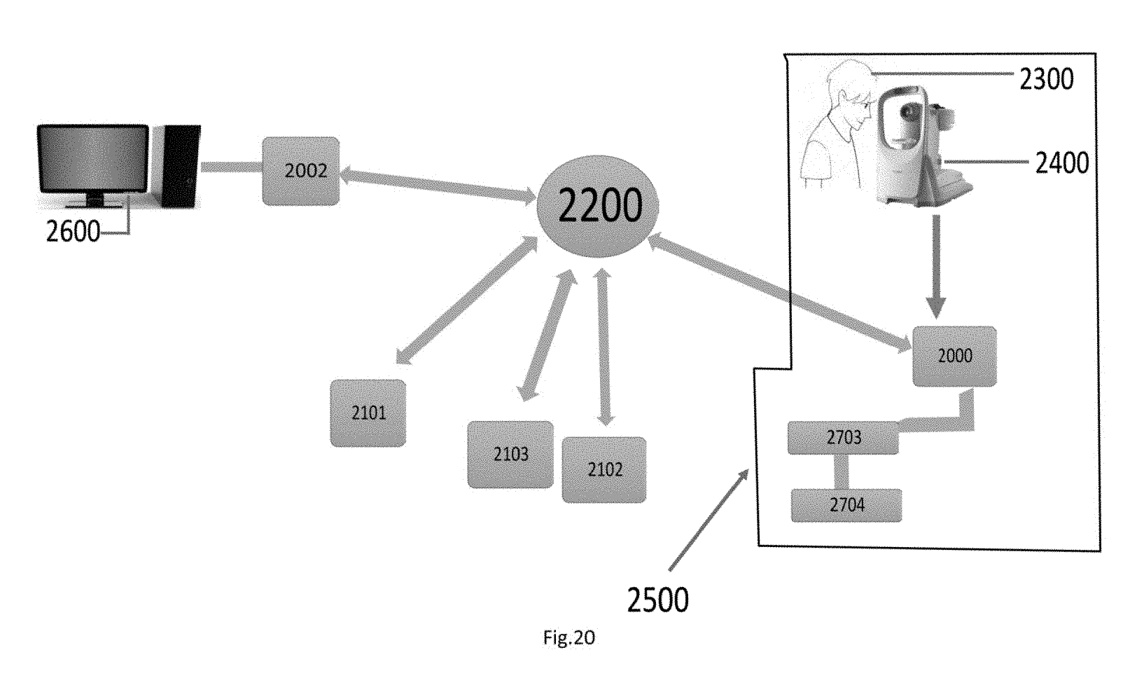

The optic disc is generally the brightest region in the retinal image. However, due to the presence of retinal pathologies such as drusen and geographic atrophy, or imaging artifacts such as illuminance and abrupt noise, it is not always the brightest object in the image. Therefore, finding brightest object may not always be successful to retrieve the OD. Considering this, optic disc detection is made by finding potential OD centers (step 803) based on intensity, OD anatomical features (i.e., circular or oval shapes) and by using blood vessel structural information as shown in FIG. 19. The OD circular edge 1901 is computed, and shape is analyzed through Hough transformation. The optic disc center is the location where the retinal blood vessels enter and exit the retina. Many branch points exist in the OD region. Thus, in the post-processing step, the branch points, and vessel centerline to determine the true OD center is considered. The method for optic disc detection is shown in FIG. 8.

### Procedure for Finding OD Center:

According to an example embodiment, the basic steps of our OD detection method is as follows:
1. Pre-processing (step 802)
   a. Calculate image calibration factor (image pixel to pixel distance in microns)
   b. Extract green color channel
   c. Create a mask for retinal region
2. Vessel Segmentation and skeletonization
3. Finding potential optic disc centers (step 803)
   a. Compute approximate OD area in pixel-number
   b. Histogram analysis and threshold image
   c. Region growing and merge close potential OD centers
4. Analyzing potential OD centers
   a. Shift potential optic disc centers
   b. Analyze each shifted center by looking at the characteristics of the surrounding blood vessels, which include:
      i. Finding the number of vessel segments around the OD center
      ii. Measuring the width and slope of these vessel segments
      iii. Extrapolating these vessel segments towards the shifted center
   c. Obtain the information of the shifted OD center coordinates, the number of vessel segments surrounding the center, and the summed variance of the x and y coordinates of the extrapolated points and store in a matrix
   d. Create a database with columns representing a shifted center and its rows containing the width information of all segments corresponding to the shifted center
5. Compute the approximate OD center (step 805)
   a. Filter out shifted centers that have fewer vessel segments than the median number of vessel segments of all shifted centers
   b. Select top quarter of shifted centers with the most votes on width
   c. Sort filtered list based on the average of the distance of the extrapolated points to the shifted center
   d. Average the shifted center coordinates of the first quarter of the filtered list (i.e. The ones with the lowest variances) to get an approximate OD center
6. Perform Canny edge detection (806) and Determine the OD center using the circular Hough transformation (807)
7. Post-processing (808)
   a. Redefine the exact OD (809) center by averaging branch point coordinates.

### Algorithm 1: Optic Disc Center Detection

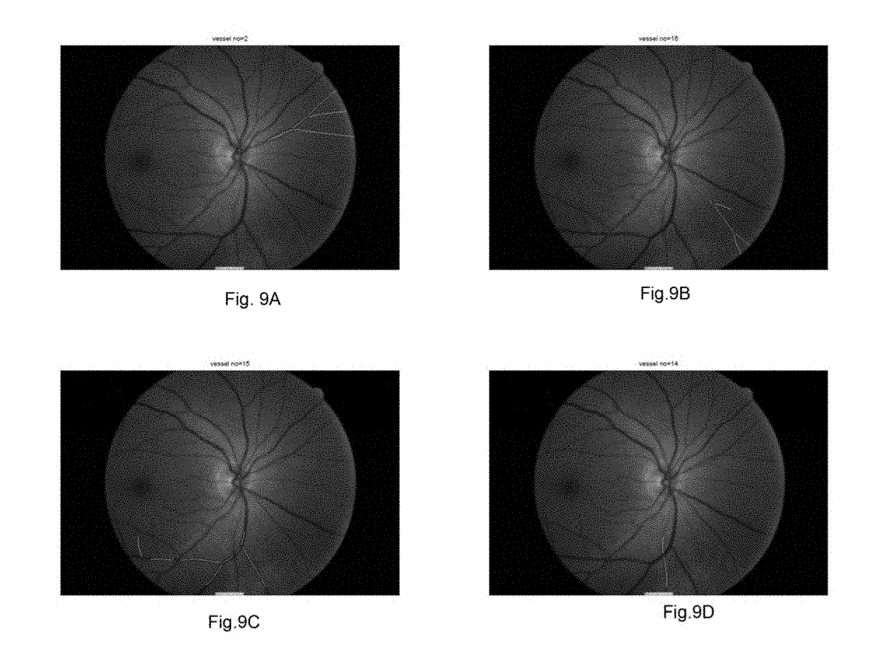

FIGS. 9A-9D show the vessel crossover mapping through individual vessel centerline identification according to an example embodiment.

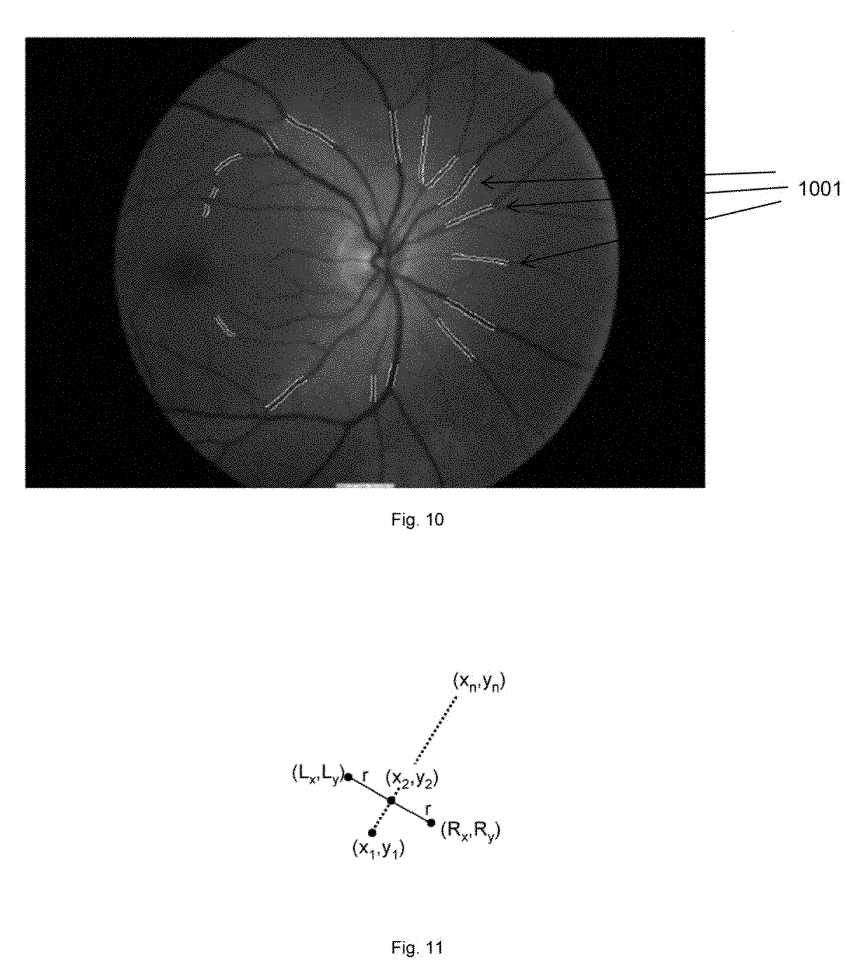

FIG. 10 shows the vessel edge or boundary 1001 in zone B area according to an example embodiment.

FIG. 11 shows the mapping of the points on left side (Lₓ;L_{y}) and right side (Rₓ;R_{y}) of an edge pixel (x₂; y₂) according to an example embodiment.

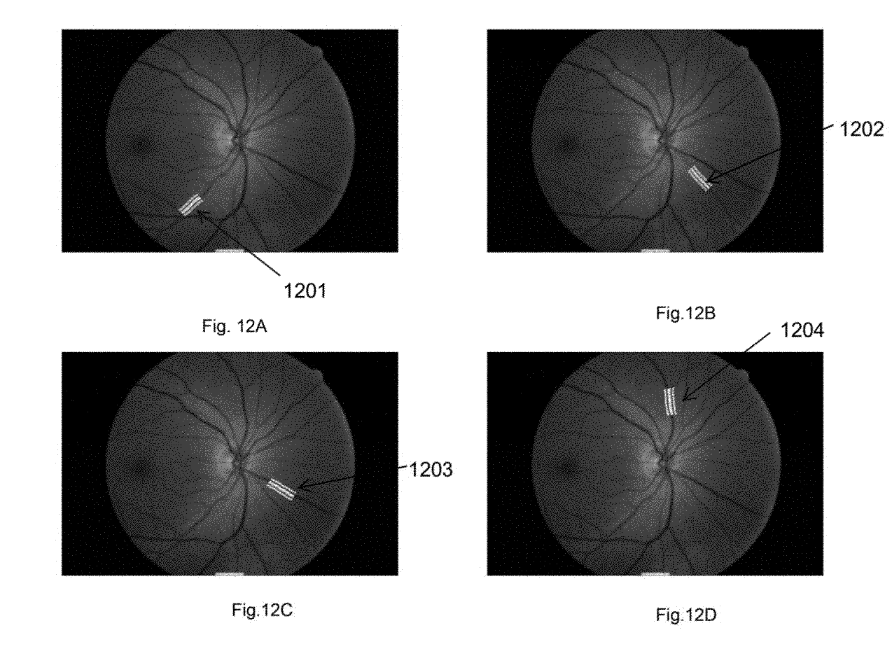

FIGS. 12A-12D show the mapping of the vessel and background pixels for intensity profiling 1201, 1202, 1203, and 1204 according to an example embodiment.

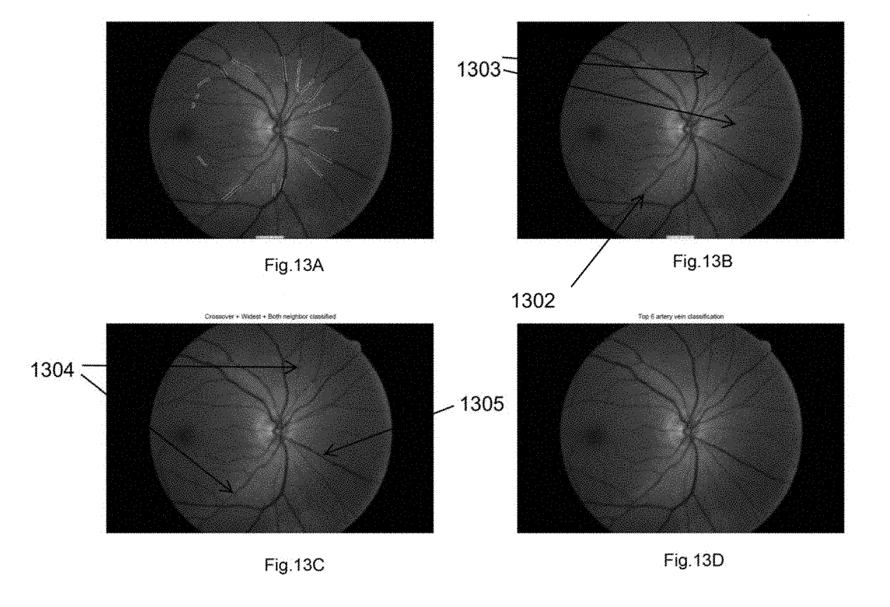

FIG. 13A shows all vessel boundary according to an example embodiment. FIG. 13B shows the classified arteries 1302 (red) and veins 1303 (blue) based on vessel crossover only (green as unclassified) according to an example embodiment. FIG. 13C shows the classified arteries 1304 and veins 1305 based on the widest vessel and classified neighbors) according to an example embodiment. FIG. 13D shows all major vessels classified as arteries and veins according to an example embodiment.

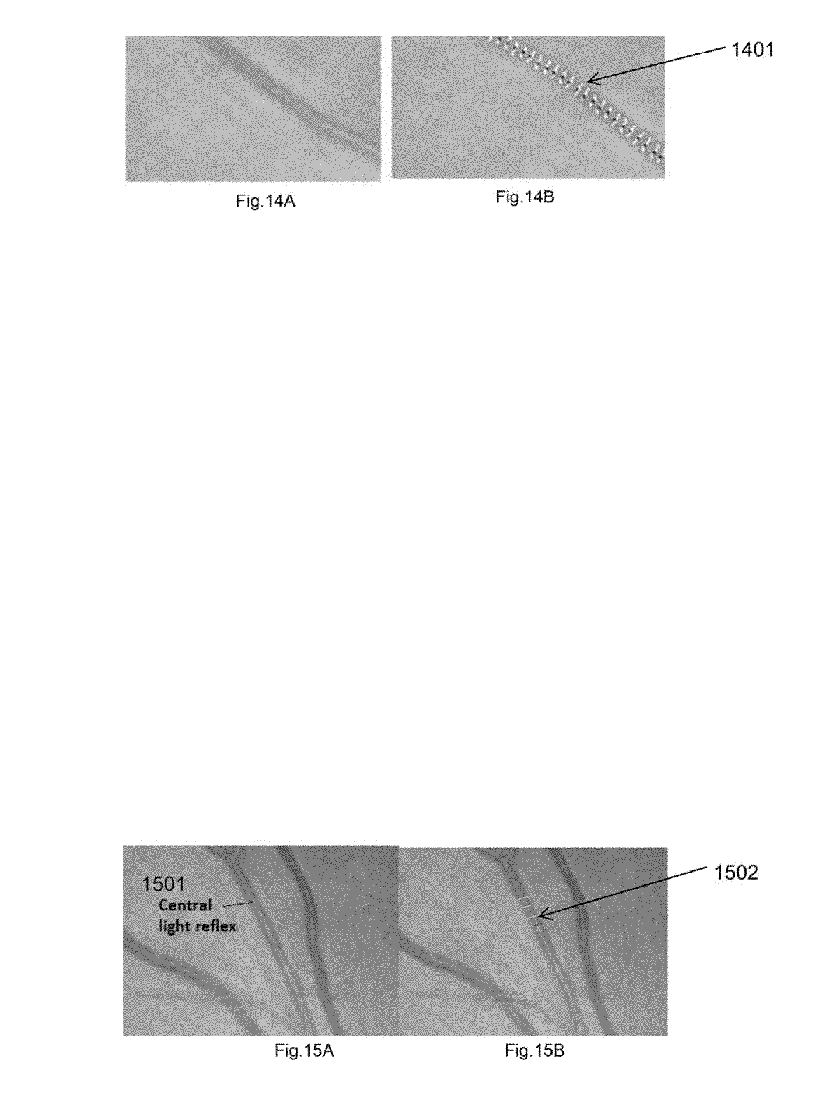

FIG. 14 shows a cropped image showing blood vessel (left) and its measured cross-sectional width 1401 (right) according to an example embodiment. FIG. 15A shows the central light reflex 1501 in an artery according to an example embodiment. FIG. 15B shows the computed widths of central light reflex 1502 (in blue) and vessel widths (in white) according to an example embodiment.

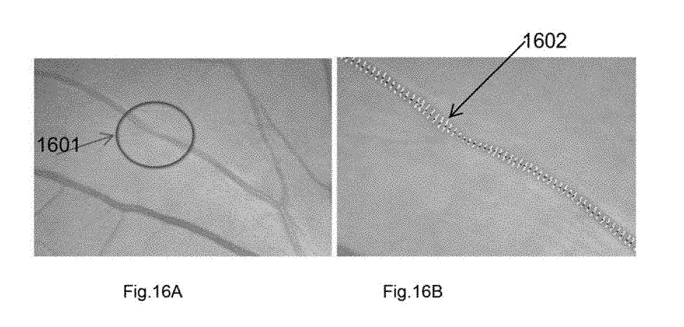

FIG. 16A shows a presence of focal narrowing 1601 in an artery according to an example embodiment. FIG. 16B shows the measured widths 1602 for the segments for finding the focal arteriolar narrowing location and quantification according to an example embodiment.

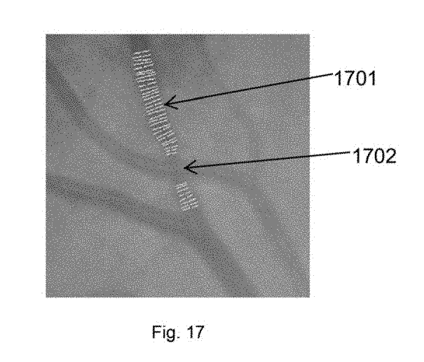

FIG. 17 shows the vein widths 7101 (white lines) for AV nicking 1702 quantification according to an example embodiment.

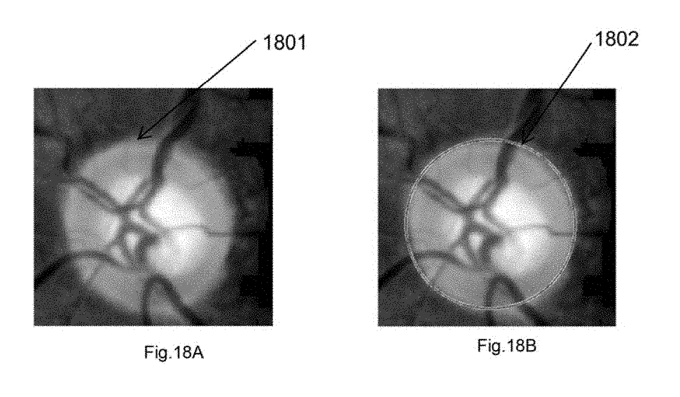

FIG. 18A shows a potential Optic Disc region 1801 according to an example embodiment. FIG. 18B shows the detected circular region 1802 of the optic disc after applying Hough transformation according to an example embodiment. FIG. 19A shows a retinal color image according to an example embodiment. FIG. 19B shows the optic disc boundary marked as red 1901 and cup boundary 1902 marked as blue according to an example embodiment.

The feature computation depends on the methods such as vessel segmentation, individual vessel segment generation, and artery-vein classification. The overall method is shown in FIG. 4 according to an example embodiment.

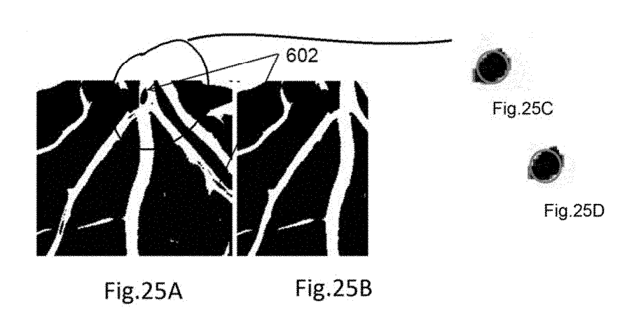

FIG. 25 shows feature (such as area) comparison associated with a central light reflex according to an example embodiment. In an example embodiment, such comparison is made based on pixel numbers in the identified features.

The vessel segmentation method aims to extract the vascular network from the background. Retinal blood vessels appear as dark and long-shaped objects on top of the retinal wall, i.e., the background. The width of the blood vessels can vary horn small microns to 200/300 microns. They have same texture and color with varying contrast and intensity distributed in the retinal image. Therefore, applying texture feature analysis is effective for producing good results on the extraction of blood vessels.

In an example embodiment, the present disclosure provides new technique which is capable of detecting blood vessel with high accuracy. The methods are automatic and are achieved by analyzing normalized intensity and texture information.

In an example embodiment, the retinal images may be obtained from any suitable source such as a fundus retinal camera 400, a database of retinal images or the like. One example of a suitable fundus retinal camera is a Canon D-60 digital fundus camera.

In some embodiments, the retinal image is received by the methods of the disclosure. In other embodiments, the retinal image is obtained as part of the methods of the disclosure. The present disclosure uses the Gabor filter to analyze the texture and normalized intensity values as features for vessel segmentation. The unsupervised clustering algorithm, i.e., Otsu's method is applied to segment the blood vessel pixel. Following this, seeded region growing operation for vessel pixel grouping and individual vessel identification is carried out.

### Features for Vessel Segmentation

In an example embodiment, the normalized intensity image bank of Gabor filters with varying angles from 0 to 180 degree (15 degrees interval) and wavelengths 5, 7 and 9 are applied and maximum response for each of the pixels is selected for clustering by Otsu's method. In another example embodiment, the wavelength can be selected anywhere between 3 to 20. The Gabor filter is applied in the frequency domain. The wavelength defines the window size to apply the mask/filter (i.e., taking the pixels from the window).

### Clustering by Otsu's Method

In an example embodiment, thresholding based pixel clustering method is applied which is based on the well-known Otsu's thresholding method. To identify the pixel clusters adequately, an algorithm is used to search for an optimal threshold level using discriminant analysis, where zero-th and first-order cumulative moments of the color histogram are calculated and used to define a measure of separability between the clusters. An optimal threshold level separating the two clusters is achieved when the within-cluster variance is minimal. The within-cluster variance is defined as a weighted sum of variances of the two clusters.

### Vessel Segmented Image

According to an example embodiment, once the clustering is completed the labeled pixels are converted (or transformed) for vessels into the binary vessel segmented image as shown in FIG. 5B.

### Post Processing, and Vessel Segmentation and Centerline Detection

According to an example embodiment, a cylindrical or tube-shaped region, 602 is identified. Such regions are the result of the presence of central light reflex in the arteries and veins. Then, a closed contour 601 representing the shaped region is identified. In an example embodiment, traverse through the vessel pixel boundary in the segmented image by region growing operation is performed to generate the closed contour. Within, this boundary, black pixels and regions which are dark, i.e., hole are also identified. Cylindrical or tube-shaped regions, 602 which are empty or zero pixel value (as shown in FIG. 6B) are identified. These shapes are 2-D and can be represented using a complex 1-D function or real 2D function. Using the boundary and centroid, the shape of the identified central reflex regions is determined. The region, its centroid, and boundary points are shown in FIG. 6. In the first variation, the values of the 1-D function for circular or oval shapes are equal to distances between the centroid and boundary points. Boundary points (A0-A4) are selected so that the central angles are equal. The distance between the subsequent boundary points for the 1-D complex function values is determined and the ratio of the minimum to the maximum radius is computed to determine the shape as a cylinder. Then the region filling operation is applied to find the complete set of vessel pixels. Following this, morphological skeletonization operation is applied on the segmented image to extract the vessel center lines.

In an example embodiment, the cylindrical tube-shaped region may extend throughout the vessel and morphological skeletonization operation is applied on the segmented image to extract the vessel centerlines.

In another example embodiment, the tube-shaped region 602 may be limited to various positions as shown in FIG. 6.

In an example embodiment, such tube-shaped regions 602 may be correlated to a disease.

In another example embodiment, a plurality of retinal images are taken at various times, and shaped regions and vessel center lines are identified as described above. Such identified shaped regions or features are compared for similarity. Based on the comparison, the features are related to a disease.

### Classify Vessel as Artery or Vein

For blood vessel classification, vessel centerlines are mapped and the vessels which cross each other are located. As only artery and vein cross each other, the—identification of vessels crossing each other helps to achieve higher accuracy on the classification. For crossover mapping—vessel centerline pixel position from traversing through the entire vessels is obtained.

### Vessel Crossing Mapping by Detecting Bifurcation and Branch Point

Vessel Crossover mapping is performed by mapping individual vessel centerline and finding the common point between vessel centerlines. The vessel centerline mapping performs centerline pixel tracking from outside optic disc and up to the end of the vessels. This method is subdivided into bifurcation and crossover point detection, vessel segment generation, vessel centerline mapping and vessel crossover or common point computation.

The centerline point mapping starts around the optic disc boundary. The method computes a circular path through the 1× disc-diameter and picks the vessel centerline points in this path. From each vessel start point, a region growing process starts for tracking the vessel centerline with the direction away from OD center. The method checks if there are more than two pixels within its 5×5 window. For a continuous vessel, the number of the pixel should be two. For crossover or branch point it should be more than two. If the number is three then the vessel segments starting points, and all pixels are mapped. Then, the vessel-segments information is inserted with its parent vessel segment information. If more than 3 pixel exist, mapping is done through the vessel segment which has a similar slope value i.e., nearest slope value. The vessel segment information is mapped as follows: segmentInformation=[segmentInformation; size(segmentInformation,1)+1 segmentPixels bifurcationPoint branchStartPixels parentSegNo];

Once a vessel is mapped then the method looks for the next vessel starting point in the circular path and so on.

FIG. 9 shows some of the mapped vessel centerlines. Once the vessel centerlines are mapped, the common pixels between the vessels are determined to identify which vessels cross each other.

Once the vessels are mapped, and the individual vessel-segments belonging to this vessel are detected. For individual vessel-segments, the vessel are split into different segments based on vessel branch point. So, one vessel-segment is from one branch point to next branch point for the vessel.

For each vessel, the crossover is mapped and based on the intensity and width level—the artery and vein are classified. For vessel classification, each vessel boundary is computed to compute pixels' intensity from inside and outside of any vessel. Each of the vessel-segments' edges are determined from vessel centerline and canny edge detection.

Vessel Edge or Boundary Mapping

For individual vessel centerline, the vessel area is selected by cropping a square shaped region. Then vessel edges are identified. For edge detection, the image is processed through the following.
Canny edge detection and the threshold is set to select a maximum number of edges.
Edge thresholding is set to select 5-10% pixels as edge pixels. With varying the threshold number, the edges are confirmed that that they are parallel to the centerline and approximately equal in length.
Within the presence of central light reflex, the maximum of four edges (two vessel edge and two central reflex edges) may be used.
Once the edges are determined, the segmented image is checked to determine if there is any presence of central light reflex. If so, the edges are considered for central light reflex.
Finally, the vessel edges that are equal distance from the centerline and have a darker pixel in the inner boundary are selected. Such edges are shown in FIG. 10.

The zone B area is considered for the vessel boundary computation and intensity matrix generation because the vessels are wider and straight in this region, and intensity distribution is also even.

### Vessel Color and Intensity Matrix Generation

For each vessel's color and intensity profile generation, the pixels from the vessel and background area are mapped. For vessel pixel mapping, first, the vessel boundary or edge are mapped and then intensity values of pixels from both sides of the vessel centerlines are determined. For this, each of the centerline pixels is considered to obtain pixels' locations on the left and the right sides of the centerline which are at a certain normal distance from this centerline pixel (FIG. 11). Each pixel and another short-distance pixel in the centerline are considered as the line end-points. The slope and the actual direction of the line are computed to find the points on both sides of the current centerline pixel. Considering (x₁, y₁) and (x₂, y₂) are two points on the edge which are considered as line end-points. The left side point (Lₓ,L_{y}) and right side point (Rₓ,R_{y}) for (x₂, y₂) are computed as follows:

Lₓ=y₂−r*sin(θ+π/2)

L_{y}=x₂+r*cos(θ+π/2)

Rₓ=y₂−r*sin(θ+3*π/2)

R_{y}=x₂+r*cos(θ+3*π/2)

where r is the normal distance from the point (x₂, y₂) and θ is the actual angle in the image which is computed from the slope and direction of the line considering two points (x₁, y₁) and (x₂, y₂), and (x₂, y₂) is the further from the OD. After computing the pixel positions on both sides of each of the centerlines points, the mean intensity levels within 3×3 windows for these positions in the image are obtained. For this, the color channel and normalized red, green and blue values are considered. Vessel color matrix mapped pixel positions (1201, 1202) in an image are shown in FIG. 12.

### Vessel Location Mapping

Each vessel position is mapped to obtain neighboring vessel information and compare the intensity matrix between the neighboring vessels. For positional information, the OD center and vessel start point angle are considered. Once all the vessels positional information is collected, the vessel angular positions are sorted and each vessel position is assigned from low to higher angle.

### Binary Classification

Two classes are applied; for labeling the blood vessels: the brighter or higher intensify vessel is artery and the darker vessel is vein. Once the intensify matrix is computed for each vessel a rule is applied based on a technique to classify them. The rules are as follows: First the wider and darker vessel is identified as a vein and the brighter vessel is identified as an artery for any vessel pair if they cross each other. Second, if a vessel does not cross with any other vessel, this vessel is considered for intensity and width measurement. If the given vessel is brighter than two of its neighbors, then it is an artery. If it is widest within its neighboring vessels and darker than any vessels in the neighbor, it is a vein. Alternatively, intensity value within the neighboring vessels is compared with the intensity of an artery. If the intensify is closer to an artery, it is an artery, else it is a vein (FIG. 3).

Algorithm 2: Vessel crossover mapping.

```
Procedure: Vessel Connectivity mapping
Input: Centerline Image, Vessel Matrix,
Output: Connectivity
begin
    1. Find the neighboring pixels of the landmark
    2. Calculate the Euclidian distance between these neighboring pixels
    and centerline pixel
    3. Sort the pixels in ascending based on the distance and put in a
    LIST
end
begin
start the search from the first pixel in the LIST while not the target
centerline pixel is found
do
    Proceed the searching through eight neighborhood connectivity
    Each time a centerline pixel is found, a value assigned to track
    indication and check for the target pixel
if the target centerline pixel is found then
      return a value for positive connectivity and break the while loop
else if certain no of iteration reaches and target pixel not found then
      consider the next pixel from the LIST until its end and go back to
      the beginning of the current section
else if certain no of iteration reaches and target pixel not found and
      LIST is empty then
      return a value for non-connectivity and break the while loop
      else
continue search
      end
      end
end
```

The method automatically classifies retinal artery and vein which is used in the automated computation of Central Retinal Arterial Equivalent (CRAE), Central Retinal Venular Equivalent (CRVE) and Arteriolar-to-Venular Ratio (AVR) to follow Knudtson protocol. The method can also be used in automated vascular feature analysis, i.e., categorizing each of the features for artery or vein.

The feature computation techniques are described as follows:

### Artery and Vein Width or Caliber Measurement

Each of the vessel centerlines is selected for its width measurement. Centerlines are selected from:
i) Zone B area
ii) Zone C area
iii) Each vessel segment's in hierarchy (after branch point based individual segment identification)
   For individual vessel centerline, the vessel area is selected by cropping a square shaped region. Then vessel edges are identified. For edge detection, the following procedure is applied
Canny edge detection and setting the threshold to select a maximum number of edges.
Setting the edge thresholding to select 5-10% pixels as edge pixels. With varying the threshold number, edges which are parallel to the centerline and approximately equal length are selected.
Within the presence of central light reflex, the maximum of four edges are considered (two vessel edge and two central reflex edges. The presence of central light reflex is mapped based on the vessel segmented image and vessel hole identification (described in the post processing step of [016] Vessel Segmentation).
Once the edges are identified, the segmented image is checked for the presence of central light reflex. If the central light reflex is present, the edges are considered for central light reflex.
Finally, the edges as vessel edges which have equal distance from the centerline and darker pixel in the inner boundary are selected.

Once vessel edges are mapped, the edge pixels from starting (near optic disc) to the end of the edges are sorted. The distance between one edge pixel to the opposite edge pixels up are found. The shortest distance for one edge pixel to the opposite edge pixels which are the cross-sectional width caliber for each of the vessel cross-sections is calculated. FIG. 14 shows the cross-sections by white lines. Each black pixel in the vessel center is showing each cross-sectional position. The end points (Green) of the white line show the edge pixels which determine the distance or width of this cross-section.

### Quantifying Arteriolar Central Reflex

For each major vessel segments, the segmented image is checked to identify if there is any central light reflex. This is performed by using the hole's shape information in the vessel center (described is post processing step of [016] Vessel Segmentation). If central light reflex is present, edges and width of the central light reflex is computed. Central reflex and vessel edge detection follow the steps as [19]. Then, the ratio of central light reflex 1501 width, and vessel width 1502 as shown in FIG. 15 is computed.

### Focal Narrowing Quantification

For each major artery segment, the width is measured to determine if there is any focal narrowing. For this, a number of cross-sections are selected (e.g., 5, 6 or up to 20 cross-sections) and an average of cross-sectional widths is computed. Considering this cross-sectional segment, the vessel-segment is divided into a number of cross-sectional-segments. Then the ratio of the mean width of cross-sectional segments are computed. If the ratio is 0.85 or less, the cross-sections is considered as FAN (FIG. 16). If the ratio is 0.6 or less, this is considered a severe FAN (FIG. 17). If the ratio is above 0.6 and below 0.75, this is considered moderate FAN and if the ratio is above 0.75 and below 0.85, this is considered as mild FAN.

### AV Nicking Quantification

For AV nicking, the crossover points for major arteries and veins are selected. In an example embodiment, the widths for all vein widths is computed. Then the cross-sections length often segments which are in the near of crossover points selected. The mean width of the segment by finding the center cross-sections and totals 10 cross-sections around it is computed. Then, the ratio of these two groups (mean width of the AVN part near crossover part of the vessel and the mean width of the normal part of the vessel) is measured. If the ratio is 0.5 or less, this is considered a severe AVN (FIG. 17). If the ratio is between 0.5 to 0.7, this is considered moderate AVN and if the ratio is between 0.7 and 0.85, this is considered as mild AVN.

In another example embodiment, AVN ratio of 0.6 and 0.7 may be considered as moderate and AVN ratio of 0.8 and 0.85 may be considered as mild.

### Hierarchical Width-Normalized-Tortuosity

For each vessel centerline, the curvature tortuosity and simple tortuosity is computed. For curvature tortuosity, the slope difference between two pixels within 3 pixels interval/distance in the centerline is computed, the average of which is considered as the curvature tortuosity. For simple tortuosity, the total number of pixels in the centerline and the Euclidian distance of the first and last pixels in the centerline is computed. Then the ratio is computed to determine the simple tortuosity.

Once the tortuosity is computed, the tortuosity is normalized with respect to the width of the vessel segment by multiplying the width and tortuosity and dividing by the maximum width of arteries and veins.

Widths within zone B and Zone C areas are represented.

### Hierarchical-Branch/Bifurcation Angle

Vessel branch angle is obtained from the children segments' centerline for each hierarchical position along with vessel number, type (artery and vein) and position. For angle computation, the two children segments' centerlines are selected. The children segments start points are considered as the line start point for slope computation. For each children segment, end point is selected from 10 pixels distance from the start point in the vessel centerline. Then the slopes of the segments are calculated separately and the angle is computed based on their slope.

The branch angle between the children vessels is represented hierarchically which corresponds to the parent vessel. The zone B and Zone C information are also used separately to represent the computed branch angles.

### OD Radius and OD Cup-Disc Ratio Computation

The automatic cup-disc ratio is computed from optic disc area, and boundary information described earlier in [017]. In the optic disc region, the pixels are clustered by Otsu's clustering method. 3 clusters are assigned, and the highest (cup) and 2^{nd} top pixel (disc) and the lowest category pixel values are assigned as blood vessel pixels. Then the cup area is computed from the pixel number. Based on the center and radius the disc area is computed with following the circle equation. The ratio of the cup and disc pixels is obtained as a cup-disc ratio.

### An Online Image Grading Platform and Automated Report Generation

In a further implementation, an online grading system 2000 configured to facilitate image transfer from a remote capturing system and configured to grading image and finally reporting is provided. The online system generates reports based on cutoff values and alerts the remote health care worker to further advise to the patient. At first, remote health care provider uses the retinal image collection system/interface and an eye camera 2400 to take fundus images from a patient. Then the healthcare provider uses a web browser or an application to login into a server system comprising processor 2601 and memory 2602 and inputs the patient data and associated disease history and upload the image(s) into the server system. The server side module/program stores the patient information into a server's database and automatically calls a software to grade the image. An image grader will be automatically assigned based on the availability to decide about the grading quality, check and finalize the feature gradings. Then a decision support system (i.e., software module 2603) automatically examines the quantified feature values and compares the cutoff values associated with specific diseases. For example, the system checks the vein width and compares the values for the risk of cardio vascular diseases. Then, the system automatically generates a report for the healthcare worker as well as patient with recommending a referral of the patient to an ophthalmologist or an expert Doctor/Consultant.

In an example embodiment, the online system 5000 for facilitating image transfer from a remote capturing 2500 to image grading system/module 2603 and generating an alert to the remote healthcare worker stationed at 2600 to further advise to a patient. The system comprises a digital fundus camera 2400 configured to capture a retinal image; a server, comprising: a processor 2700 and a memory 2701, wherein the memory comprises a non-transitory computer-readable-medium having computer-executable instructions stored therein that, when executed by the processor, causes the processor to perform the following steps. The process 2700 first stores the retinal image and the associated patient information into the server data base 2612. The server then calls a first software module 2702 stored in the server to evaluate and grade the fundus image. The processor 2700 is further configured to select an image grader from a list of authorized image grader stored in the server to inspect and finalize features and grading the quality of the fundus image. Image graders are selected based on a location information and availability of the image graders. In response to receiving an authorization, of the fundus image by the image graders, the processor calls a second software module to automatically examine and quantify a plurality of feature values in the fundus image. In a further step, the processor calls an evaluating and grading algorithm 2603 stored in the server to compare the quantified features values with a plurality of cutoff or threshold values associated with specific diseases and generates a report recommending a referral to an expert based on the comparison.

In yet another example embodiment, a system for image capturing, uploading and processing unit 2500 comprises a digital fundus camera 2400 configured to capture a retinal image from a patient 2300 and process and send the image to a CPU including a memory 2701 and processor 2700. In an example embodiment, the memory 2701 comprises a non-transitory computer-readable-medium having computer-executable instructions stored therein that when executed by the processor, causes the processor to: pre-process the retinal image and store the pre-processed image in the memory; perform texture analysis using a Gabor filtering module;

perform Otsu's clustering to cluster or segment vessel pixels of the texture analyzed image; detect one or more central light reflexes by identifying a shaped region; and perform region filling to generate a vessel segmented image of the retinal image.

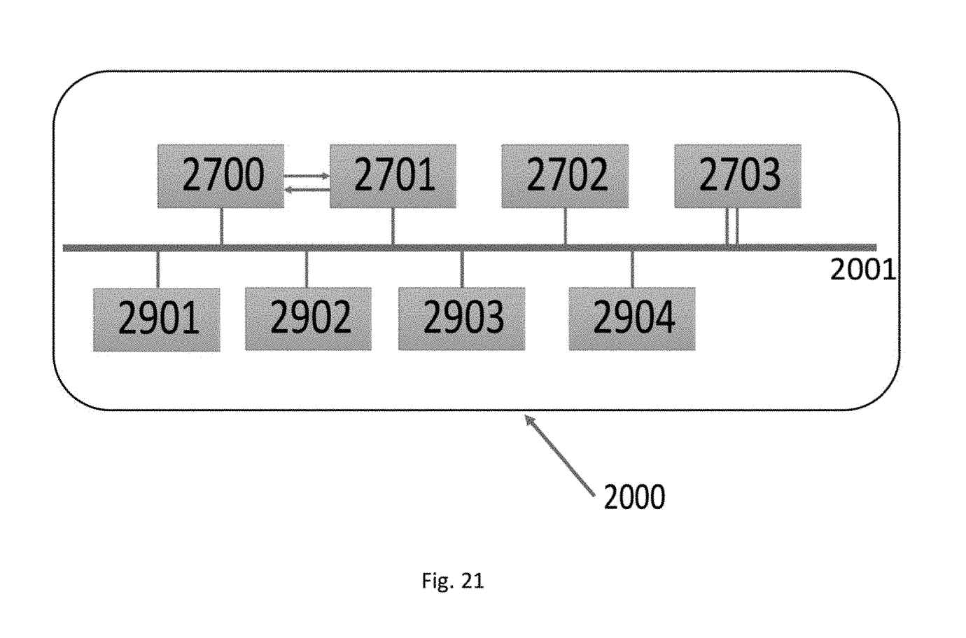

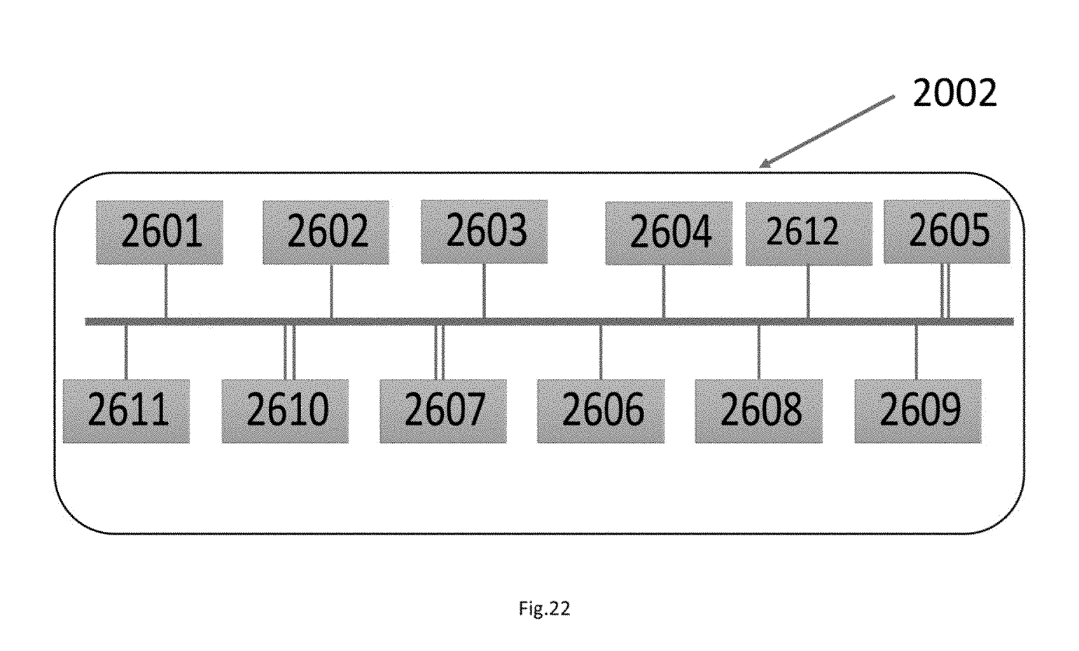

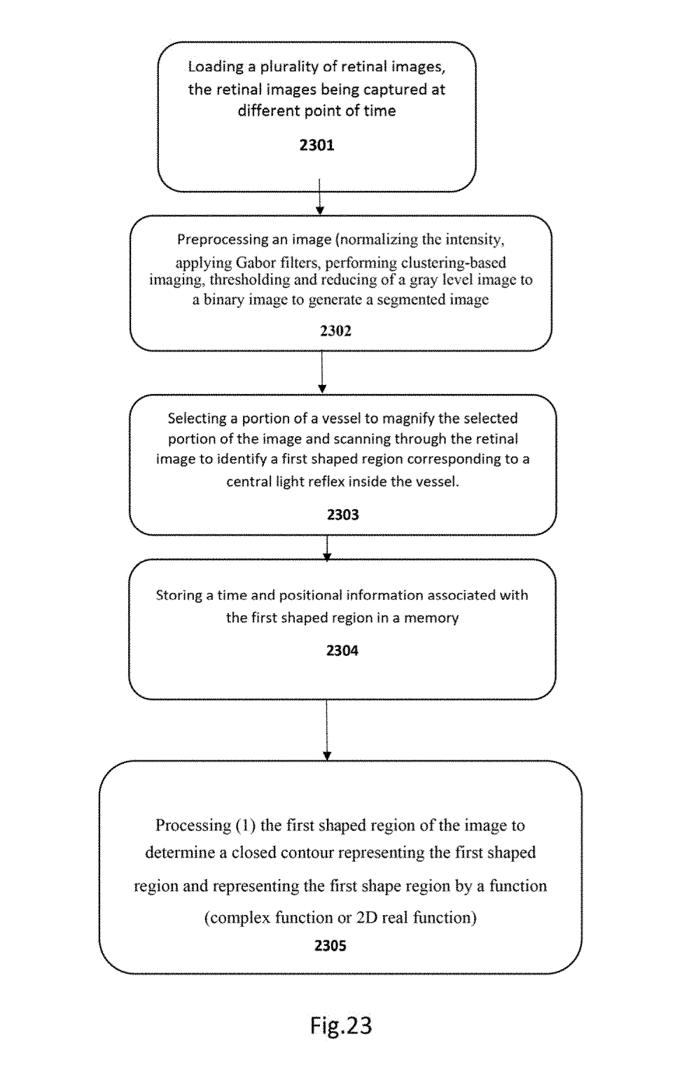

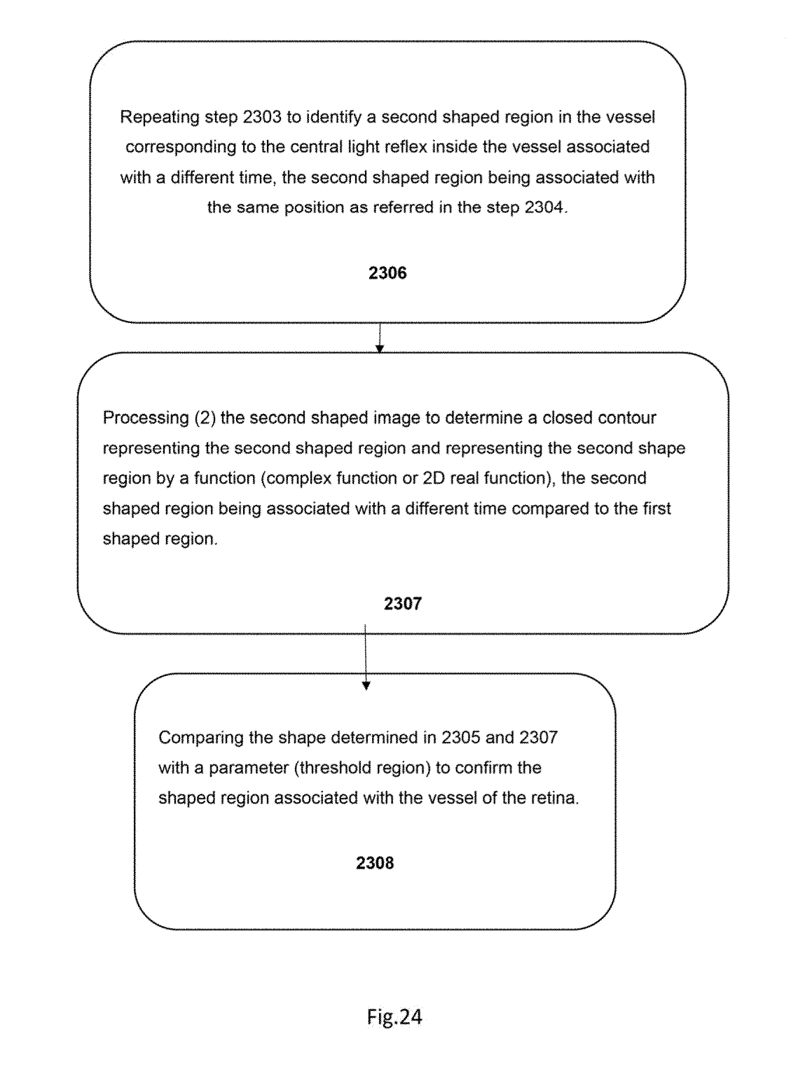

Table 1 describes various modules, systems and system components used in FIGS. 20-22.

TABLE 1

| Reference | |
|---|---|
| Numbers | Descriptions |
| 2000 | Interconnected devices at the image capturing station |
| 2001 | Interconnect architecture |
| 2700 | Processor (CPU), desktop, tablets, cell phones |
| 2701 | Memory unit |
| 2702 | Software module |
| 2703 | Touch screen/user interface |
| 2704 | Keyboard interface |
| 2901 | Wireless communication module |
| 2902 | Wired input/output port |
| 2903 | Wired input/output port to retinal camera |
| 2101 | Server in a remote location A |
| 2102 | Server in a remote location B |
| 2200 | internet |
| 2400 | Retinal camera |
| 2300 | Patient for report generation |
| 2500 | Image capturing, uploading and processing unit |
| 2002 | Interconnection at the grader station |
| 2600 | Grader station |
| 2601 | Processor (CPU), desktop, tablets, cell phones |
| 2602 | Memory - storing instructions |
| 2603 | Module comprising Evaluating and grading algorithm |
| 2604 | Grader availability, selection module and user specific |
| | authorization |
| 2605 | Memory database - storing licensed grader and login |
| | credentials |
| 2608 | Virtual Training modules |
| 2611 | Memory database storing licensee owner data |
| 2612 | Patient record database |
| 2606 | Security/encryption modules |
| 2607 | Report generating module |
| 2609 | Standardizing and reference data storing and comparing |
| | module |
| 2610 | Data backup module |

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present inventive concept have been described above about specific embodiments. These benefits and advantages, and any elements or limitations that may cause them to occur or to become more pronounced are not to be construed as critical, required, or essential features of any or all of the embodiments.

While this specification contains many specific implementation details, these should not be construed as limitations on the scope of any inventive concept of what may be claimed, but rather as descriptions of features that may be specific to particular embodiments of particular inventive concept. Certain features that are described in this specification in the context of separate embodiments can also be implemented in combination in a single embodiment. Conversely, various features that are described in the context of a single embodiment can also be implemented in multiple embodiments separately or in any suitable sub-combination.

What is claimed is:

1. A method for vessel classification, the method comprising:
pre-processing a retinal image to generate a vessel segmented image;
first processing the vessel segmented image to generate an image with a central light reflex;
identifying a cylindrical shaped region in the central light reflex;
determining a closed contour representing the cylindrical shaped region and representing the closed contour by a function;
computing a ratio of a minimum and maximum radius of the cylindrical shape of the central light reflex to determine an average shape of the cylinder associated with the central light reflex by using the function;
performing region filling for filling at least a hole in vessel center, the hole being formed due to central light reflex;
second processing the image to apply a morphological skeletonization operation to generate vessel, centerlines and segmented vascular network of the retinal image.

2. The method according to claim 1, wherein identifying the cylindrical shaped region further includes region growing by a grouping of pixel using seeded region growing technique.

3. The method according to claim 1, wherein the retinal image is at least one of a color image, grayscale image, infrared image, auto-fluorescence image, green channel image, red-free image and a combination thereof.

4. The method according to claim 1, further comprising classifying the arteries and veins based on parameters associated with the vessel-segments and hierarchical information.

5. The method according to claim 1, further comprising classifying the arteries or veins based on a combination of a first parameter and a second parameter.

6. The method according to claim 1, further comprising computing the ratio of central light reflex width and vessel width.

7. The method according to claim 4, wherein the first parameter is associated with at least one of vessel width, central reflex width, vessel color and intensity matrix, and vessel angular positional information and a second parameter is associated with the retinal image.

8. The method according to claim 1, further comprising:
selecting a portion of a vessel to magnify the selected portion of the image and scanning through the retinal image to identify a first shaped region corresponding to a central light reflex inside the vessel;
storing a time and a first positional information of first shaped region associated with the central light reflex in a memory;
identifying a second shaped region associated with the central light reflex inside the vessel associated with a different time and corresponding to the first positional information; and
comparing the first shaped region and second shaped region to confirm the shaped region associated with the central light reflex.

9. The method according to claim 8, wherein comparing includes comparison of the first shaped region and second shaped region based on an area overlap between the two shapes.

10. A method of optic disc detection from a retinal image, the method comprising:
processing the retinal image to generate a vessel segmented image to identify blood vessels;
determining approximate optic disc centers based on a first information associated with the retinal image and the first set of a parameter associated with the vessels;
processing the retinal image to determine a plurality of shifted optic disc centers from among the approximate optic disc centers;
filtering the shifted optic disc centers based on a criterion and selecting a number of optic disc centers and determining an average of the selected optic disc centers to obtain a first optic disc center;
performing Hough transformation using the first optic disc center to determine a first optic disc; and
determining a second set of parameters associated the first optic disc center and the first optic disc; and
using a combination of the second set of parameters to Identify a true optic disc (OD).

11. The method of claim 10, wherein identifying of the true optic disc (OD) include identifying the center and radius of the true optic disc (OD).

12. The method of claim 10, wherein a cup-disc area is determined by applying Otsu's clustering method to a cluster the pixels within an area/boundary of the true optic disc (OD).

13. The method of claim 10, wherein the first information includes optic disc anatomical features, and the first set of parameters includes parameters associated with intensity, vessel center line, blood vessel structures, width and slope of the vessel segments, and a number of vessel segments surrounding an optic disc of the approximate optic disc center.

14. The method of claim 10, wherein the second set of parameters are the parameters associated with vessel centerline, vessel segments alignment, length, width and slope of the vessel segments, vessel segment numbers and vessel branch-point density of the first optic disc.

15. The method of claim 12, wherein a cup-disc ratio is determined by using a total number of pixels in a cup of the true optic disc and disc region of the true optic disc.

16. The method of claim 15, wherein, the retinal image includes at least one of the color image, grayscale image, infrared image, auto-fluorescence image, green channel image, red-free and a combination thereof.

17. A method for retinal blood vessel feature quantification, the method comprising:
processing a retinal image to generate vessel centerline;
generating a vessel segmented image of a retinal image to identify blood vessels; and
further comprising classifying the arteries or veins based on a combination of a first parameter and a second parameter.

18. The method according to claim 17, further comprising:
selecting a vessel area by cropping a square shaped region;
performing canny edge detection and setting a threshold to select a set of pixels in each edge;
determining a distance between one edge pixel to the opposite edge pixel for all pixels based on a position or index of the edge pixels; and
finding a shortest distance among the determined distances to determine the vessel width for a cross-section in the selected vessel area.

19. The method according to claim 18, further comprising selecting a number of cross-sections in a vein or artery along a narrow region and in a wide region adjacent to the narrow region;
determining a ratio of the cross-sectional width between the narrow region, and wide region; and
comparing the ratio with a threshold value to quantify a focal narrowing (FAN) of the vessel.

20. The method according to claim 17, further comprising, for each vessel centerline,
computing the curvature tortuosity and simple tortuosity; and
normalizing the tortuosity with respect to the width of the vessel segment by multiplying the width and tortuosity and dividing by the maximum width of arteries and veins.

21. The method according to claim 17, further comprising processing the retinal image to generate the intensity matrix of the vessel.
a parameter associated with the vessels; and
quantifying AV nicking by,
selecting a crossover point for an artery and vein segment,
determining a mean width of vein segments away from the crossover point;
determining a mean width of vein segments close to the cross-over point;
computing a ratio of the mean width of the segments close to the cross-over point and away from the cross-over point; and
comparing the ratio with a threshold value to identify the quantified AV nicking.

22. The method of claim 21, wherein classifying arteries and veins using vessel segments' slope, colour, central reflex, parameter, intensity of the blood vessels and a combination thereof.

23. A computer implemented system for vessel segmentation of a retinal image, the system comprising:
a processor and a memory, wherein the memory comprises a non-transitory computer-readable-medium having computer-executable instructions stored therein that, when executed by the processor, cause the processor to:
pre-process the retinal image and store the pre-processed image in the memory;
perform texture analysis using a Gabor filtering module;
perform Otsu's clustering to cluster or segment vessel pixels of the texture analysed image;
detect one or more central light reflexes by identifying a shaped region; and
perform region filling to generate a vessel segmented image of the retinal image.
